(19) United States
(12) Patent Application Publication
(10) Pub. No.: US 2025/0049992 A1
(43) Pub. Date: Feb. 13, 2025

(54) Bone tissue regeneration product AND method for repairing bone defect

(71) Applicant: Shanghai Ninth People's Hospital, Shanghai JiaoTong University School of Medicine, Shanghai (CN)

(72) Inventors: Xinquan JIANG, Shanghai (CN); Jin WEN, Shanghai (CN); Yuwei DENG, Shanghai (CN); Ruixue JIANG, Shanghai (CN); Xiao WANG, Shanghai (CN); Qianju WU, Shanghai (CN); Ran YAN, Shanghai (CN)

(73) Assignee: Shanghai Ninth People's Hospital, Shanghai JiaoTong University School of Medicine, Shanghai (CN)

(21) Appl. No.: 18/783,570

(22) Filed: Jul. 25, 2024

Related U.S. Application Data
(63) Continuation-in-part of application No. 17/376,191, filed on Jul. 15, 2021.

(30) Foreign Application Priority Data
Mar. 22, 2021 (CN) 2021103032733

(51) Int. Cl. A61L 27/54; A61K 38/18

(52) U.S. Cl. CPC A61L 27/54; A61K 38/18; A61L 2430/02

(57) ABSTRACT

A bone tissue regeneration product, a method for preparing the bone tissue regeneration product and, and a method for repairing a bone defect. The new use of IGSF10 of the present disclosure can reduce the effective concentration of BMP without obvious adverse reactions, thereby exploring a method to promote bone tissue regeneration and providing new ideas for the treatment of bone defects. The present disclosure is capable of promoting non-bone related cells to produce bones; the method of the present disclosure is also capable of inhibiting the bone loss in inflammatory environments.

### CROSS REFERENCE TO RELATED APPLICATIONS

This application is a continuation-in-part of U.S. patent application Ser. No. 17/376,191 filed Jul. 15, 2021, the entire disclosure of which is hereby incorporated by reference.

### FIELD OF THE INVENTION

The present disclosure relates to the field of medicines, in particular, to the use of IGSF10 in the preparation of bone tissue regeneration products.

### BACKGROUND OF THE INVENTION

The regeneration of defective bones by tissue engineering is a hot research topic. Growth factors play an important role in regulating cell proliferation and migration, and extracellular matrix synthesis, which can effectively promote wound healing and promote the growth of bone tissue. Growth factors commonly used in clinic application include bone morphogenetic proteins (BMPs) that promote bone regeneration, platelet-derived growth factor (PDGF), enamel matrix protein (EMP), and amelogenin, et al. The BMP family promotes the differentiation and maturation of stem cells and plays an important role especially in bone and cartilage formation. However, the above-mentioned factors are often accompanied with adverse reactions such as swelling and ectopic mineralization during clinical applications. Therefore, the finding of alternative or synergistic growth factors for realizing bone tissue repair has become a current research hotspot. Additionally, bone loss caused by inflammation in diseases such as periodontitis is also an urgent issue that needs to be addressed. Inflammatory environments lead to the release of inflammatory cytokines, resulting in an imbalance in bone metabolism. Therapies interfering with inflammation also affect inflammatory bone loss. Therefore, inflammation is closely related to bone loss, and the inflammation must be effectively suppressed before bone formation.

### SUMMARY OF THE INVENTION

The present disclosure provides the use of IGSF10 in the preparation of bone tissue regeneration products, to solve the problems in the traditional technology.

The present disclosure provides the use of IGSF10 in the regeneration of bone tissues.

Preferably, the use is the use of IGSF10 in combination with one or more of bone morphogenetic protein (BMP), platelet-derived growth factor (PDGF), enamel matrix protein (EMP) and amelogenin in the regeneration of bone tissues.

The present disclosure further provides the use of IGSF10 in the preparation of bone tissue regeneration products.

Preferably, the use is the use of IGSF10 in combination with one or more of bone morphogenetic proteins (BMPs), platelet-derived growth factor (PDGF), enamel matrix protein (EMP) and amelogenin in the preparation of a product for promoting bone tissue regeneration.

The present disclosure further provides a bone tissue regeneration product, which includes a bone repair material and one or more growth factors loaded onto the bone repair material, wherein the one or more growth factors include growth factor IGSF10.

Preferably, the amino acid sequence of the growth factor IGSF10 is set forth in SEQ ID NO. 1.

Preferably, the one or more growth factors in the product further include one or more of BMP, PDGF, EMP, and amelogenin.

Preferably, the one or more growth factors in the product further include BMP.

Preferably, the bone tissue regeneration product includes a periodontal bone defect repair product, a jaw bone defect repair product, a skull defect repair product, and/or a long bone defect repair product.

Preferably, the bone tissue regeneration product is drug, health care product, food, or consumable.

Preferably, the bone repair material is a hydroxyapatite scaffold, a calcium phosphate scaffold, a hydrogel scaffold, or a bioglass scaffold.

The present disclosure further provides a method for preparing a bone tissue regeneration product, comprising: adding one or more growth factors to a bone repair material; and allowing the bone repair material added with the one or more growth factors to stand for 0.5-2.5 hours at a constant temperature, wherein the one or more growth factors include IGSF10.

Preferably, the bone repair material added with the one or more growth factors stands for 0.5-2.5 hours at a constant temperature under negative pressure: preferably, the bone repair material added with the one or more growth factors stands for 0.5-2.5 hours at a constant temperature under vacuum.

Preferably, the method further includes one or more of the following:
1) the one or more growth factors further include one or more of BMP, PDGF, EMP and amelogenin;
2) the constant temperature is 25-37° C., 4° C. or −20° C.;
3) freeze-drying the bone repair material added with the growth factors;
4) allowing the bone repair material added with the one or more growth factors to stand at a constant temperature under vacuum or negative pressure;
5) the ratio of the total mass of the one or more growth factors to the total mass of the bone repair material is 0.1:1˜15:1.

In some embodiments, the constant temperature is 25-37° C.; in some embodiments, the constant temperature is −20° C.: in some embodiments, the constant temperature is 4° C.

The present disclosure further provides a method for repairing a bone defect, including the following operations: administering the above mentioned bone tissue regeneration product to a bone defect site, wherein the bone tissue regeneration product comprises a bone repair material and one or more growth factors loaded onto the bone repair material, wherein the one or more growth factors include growth factor IGSF10.

Preferably, the bone defect site includes bone related cells and/or non-bone related cells.

Preferably, the bone defect site includes non-bone related cells and does not comprise bone related cells.

Preferably, the non-bone related cells include dental pulp cells and/or periodontal ligament cells.

Preferably, the bone defect comprises a periodontal bone defect, a jaw bone defect, a skull defect, and/or a long bone defect.

Preferably, the bone defect is a periodontal bone defect.

Preferably, the method further includes administering the bone tissue regeneration product to an inflammatory tissue microenvironment.

Preferably, the method further includes: administering the bone tissue regeneration product containing an effective amount of the one or more growth factors to a bone defect site.

As mentioned above, the use of IGSF10 of the present disclosure in the preparation of bone tissue regeneration products has the following beneficial effects: the effective concentration of BMP, PDGF, EMP or amelogenin could be reduced, and adverse reactions could be decreased, such that a method to promote bone tissue regeneration is explored, and new ideas for the treatment of bone defects are provided; the method of the present disclosure is capable of promoting non-bone related cells to produce bones: the method of the present disclosure is also capable of inhibiting the bone loss in inflammatory environments.

### BRIEF DESCRIPTION OF DRAWINGS

FIG. 1 shows the osteogenic effects of IGSF10 and BMP2 on DPC and PDLC ((A): alizarin red staining: (B) calcium ion releasing from cells detection).

FIG. 2 shows the H&E histological examination of the repair effect of IGSF10 and BMP2 in a periodontal defect model.

FIG. 3 shows the Micro-CT result of IGSF10 and BMP2 in repairing rat periodontal bone defects.

FIG. 4 shows the Micro-CT effect of IGSF10 and BMP2 in repairing skull defects in vivo.

FIG. 5 shows the results of IGSF10 and BMP2 in nude mice subcutaneous heterotopic osteogenesis experiment.

FIG. 6 shows the immunofluorescence staining results of the spread of DPCs and PDLCs treated with IGSF10.

FIG. 7 shows the western blot results of osteogenesis-related proteins and bone transcription factors in DPCs and PDLCs treated with IGSF10.

FIG. 8 shows the immunofluorescence staining results of the nuclear translocation of YAP protein in PDLCs treated with IGSF10.

FIG. 9 shows the pathway kit results of IGSF10-treated DPCs and PDLCs.

FIG. 10 shows the western blot experiment confirming that IGSF10 increases the phosphorylation of STAT1 and JNK in DPCs and PDLCs.

FIG. 11 shows the micro-CT results of femur in IGSF10 knockout mice and control mice.

FIG. 12 shows the micro-CT results of femur in IGSF10 knockout mice and control mice.

FIGS. 13A-13D show osteogenic ability of BMSCs in IGSF10 knockout mice and control mice, wherein FIG. 13A shows alizarin red staining, FIG. 13B shows ALP staining, FIG. 13C shows detection of osteogenic genes by qPCR, and FIG. 13D shows detection of osteogenic proteins by western blot.

FIG. 14 shows the micro-CT results of maxillary alveolar bone in periodontitis mice and periodontitis mice treated with IGSF10.

FIG. 15 shows the micro-CT results of maxillary alveolar bone in periodontitis mice and periodontitis mice treated with IGSF10.

FIG. 16 shows the H&E histological examination of maxillary alveolar bone in periodontitis mice and periodontitis mice treated with IGSF10.

### DETAILED DESCRIPTION OF THE INVENTION

The present disclosure is the first to provide the use of IGSF10 in the regeneration of bone tissues.

Immunoglobulin superfamily member 10 (IGSF10) belongs to the immunoglobulin superfamily and act as a growth factor. Unless otherwise specified in the present disclosure, bone tissue regeneration includes cartilage tissue regeneration and bone tissue regeneration. Regeneration refers to the repair of damaged cells or tissues by the division and proliferation of neighboring healthy tissue cells.

Specifically, the use of IGSF10 is in the regeneration of bone tissues in mammals.

The mammals are preferably rodents, artiodactyls, perissodactyls, lagomorphs, primates, or the like. The primates are preferably monkeys, apes or humans.

In an embodiment, the application is the use of IGSF10 in promoting the regeneration of bone tissues. IGSF10 can promote bone tissue regeneration by inducing and maintaining the differentiation of bone or cartilage at an early stage of development.

The bone tissue may be, for example, periodontal bone, jaw bone, skull, tibia, fibula, femur and other bone tissues of the whole body skeletal system.

In a preferred embodiment, the use is the use of IGSF10 in periodontal bone defect repair, jaw bone defect repair, skull defect repair, and/or long bone defect repair.

The IGSF10 may be a natural protein or a recombinant protein.

Further, the IGSF10 includes a protein formed by any IGSF10 sequence derived from human, murine, canine, bovine, porcine and the like. Information about the above-mentioned basic protein sequences from different sources may be retrieved from NCBI. In an embodiment, the IGSF10 is selected from human recombinant IGSF10, and the amino acid sequence is shown in SEQ ID NO. 1:

(SEQ ID NO. 1)
SAFISPQGFMAPFGSLTLNMTDQSGNEANMVCSIQKPSRTSPIAFTEEN
DYIVLNTSFSTFLVCNIDYGHIQPVWQILALYSDSPLILERSHLLSETP
Q

In a preferred embodiment, the use is the use of IGSF10 in combination with one or more of bone morphogenetic protein (BMP), platelet-derived growth factor (PDGF), enamel matrix protein (EMP), and amelogenin in the regeneration of bone tissues. The combination application can reduce the effective concentration of several other growth factors, thereby reducing adverse reactions.

In an embodiment, the BMP is BMP-2.

In an embodiment, the concentration of each growth factor may be 200-600 ng/ml when used in combination. For example, the concentration may be selected from one of the following concentration ranges: 200-250 ng/mL, 250-300 ng/ml, 300-350 ng/mL, 350-400 ng/mL, 400-450 ng/mL, 450-500 ng/mL, 500-550 ng/mL, and 550-600 ng/mL.

Those skilled in this field understand that the buffer for diluting the growth factors of IGSF10, BMPs, PDGF, EMP or amelogenin may be commonly used buffer such as normal saline, PBS, or Tris, provided that the nature of the growth factors is not changed.

The present disclosure further provides the use of IGSF10 in the preparation of bone tissue regeneration products.

Specifically, the use is the use of IGSF10 in the preparation of mammalian bone tissue regeneration products.

The mammals are preferably rodents, artiodactyls, perissodactyls, lagomorphs, primates, or the like. The primates are preferably monkeys, apes, or humans.

In an embodiment, the use is the use of IGSF10 in the preparation of a product for promoting bone tissue regeneration.

The bone tissue may be, for example, periodontal bone, jaw bone, skull, or long bone.

In a preferred embodiment, the use is the use of IGSF10 in the preparation of a product for repairing the periodontal bone defect, jaw bone defect, skull defect, and/or long bone defect. In an embodiment, the use is the use of IGSF10 in the preparation of a product for inducing and maintaining the differentiation of bone or cartilage at an early stage of development.

The IGSF10 may be a natural protein or a recombinant protein.

Further, the IGSF10 includes a protein formed by any IGSF10 sequence derived from human, murine, canine, bovine, porcine and the like. Information about the above-mentioned basic protein sequences from different sources may be retrieved from NCBI. In an embodiment, the IGSF10 is selected from human recombinant IGSF10, and the amino acid sequence is shown in SEQ ID NO. 1:

(SEQ ID NO. 1)
SAFISPQGFMAPFGSLTLNMTDQSGNEANMVCSIQKPSRTSPIAFTEEN
DYIVLNTSFSTFLVCNIDYGHIQPVWQILALYSDSPLILERSHLLSETP
Q

In an embodiment, in addition to IGSF10, the bone tissue regeneration product further includes one or more of bone morphogenetic proteins (BMPs), platelet-derived growth factor (PDGF), and enamel matrix protein (EMP). That is, in an embodiment, the use is the use of IGSF10 in combination with one or more of BMPs, PDGF, EMP and amelogenin in the preparation of a product for promoting bone tissue regeneration. The combination of the above growth factors can reduce the effective concentration of several other growth factors, thereby reducing adverse reactions.

In an embodiment, the concentration of IGSF10 and/or other growth factors may be 20-600 ng/ml when the product is used. For example, the concentration may be selected from one of the following concentration ranges: 20-50 ng/mL, 50-100 ng/mL, 100-150 ng/mL, 150-200 ng/mL, 200-250 ng/mL, 250-300 ng/ml, 300-350 ng/mL, 350-400 ng/mL, 400-450 ng/mL, 450-500 ng/mL, 500-550 ng/ml and 550-600 ng/mL.

Those skilled in this field understand that the buffer for diluting IGSF10, BMPs, PDGF, EMP or amelogenin may be a commonly used buffer such as normal saline, PBS, or Tris, provided that the nature of the growth factors is not changed.

The products include, but are not limited to, drugs, health care products, foods, and consumables. IGSF10 is the only effective component or one of the effective components of the product.

The product may be a single-component substance or a multi-component substance.

In an embodiment, the product is a drug, and the drug further includes a pharmaceutically acceptable excipient.

“Pharmaceutically acceptable” means that when the molecular entities and compositions are properly administered to animals or humans, they will not produce adverse, allergic, or other untoward reactions.

Furthermore, the pharmaceutically acceptable excipients should be compatible with the effective components, that is, able to blend with the effective components without greatly reducing the effect of the drug under normal circumstances. Specific examples of substances that may serve as pharmaceutically acceptable carriers or excipients include saccharides (such as lactose, glucose, and sucrose), starches (such as corn starch and potato starch), cellulose and derivatives thereof (such as sodium methyl cellulose, ethyl cellulose and methyl cellulose), tragacanth powder, malt, gelatin, talc, solid lubricants (such as stearic acid and magnesium stearate), calcium sulfate, vegetable oils (such as peanut oil, cottonseed oil, sesame oil, olive oil, corn oil and cocoa butter), polyols (such as propylene glycol, glycerol, sorbitol, mannitol and polyethylene glycol), alginic acids, emulsifiers (such as Tween), wetting agents (such as sodium lauryl sulfate), coloring agents, flavoring agents, tableting agents, stabilizers, antioxidants, preservatives, pyrogen-free water, isotonic saline solution, and phosphate buffer. The above substances are used as needed, to increase the stability of the formulation, help improve the activity or bio-availability of the formulation, or produce an acceptable mouthfeel or odor in the case of oral administration.

In another embodiment, the product is a consumable.

Specifically, the consumable may be constructed by loading IGSF10 alone or in combination with other growth factors onto a certain carrier. For example, IGSF10 and/or one or more of BMPs, PDGF, EMP and amelogenin are loaded onto the bone repair material to form the consumable. The bone repair material, as a bridge connecting seed cells and regenerated tissues, has a structure and function similar to those of natural bones and contains material with osteoinductive activity. The bone repair material may be, but is not limited to a hydroxyapatite scaffold, a calcium phosphate scaffold, a hydrogel scaffold or a bioglass scaffold.

The consumables may be prepared by loading growth factors onto the bone repair material in advance. Or, the consumables may be prepared on the spot, that is, the growth factors and the bone repair material are stored separately, and then the bone repair material is loaded with the growth factors during when application is started. The loading method may be: slowly adding the growth factors onto the bone repair material dropwise, transferring the bone repair material added with the growth factors to a constant temperature incubator, standing for 0.5 to 2.5 hours before use. The temperature of the constant temperature incubator is preferably 25-37° C., 4° C. or −20° C. In an embodiment, the bone repair material is a biological scaffold material. In an embodiment, the loading method includes adding the growth factors onto a biological scaffold material such as collagen and/or fibrin. In an embodiment, the loading method includes using microspheres/nanoparticles to encapsulate the growth factors. In an embodiment, the loading method includes adding the growth factors onto a hydrogel material such as polyethylene glycol or gelatin for localized controlled release of the growth factors. In an embodiment, the loading method includes freeze-drying the bone repair material added with the growth factors, and the temperature of the constant temperature incubator is preferably 4° C. or −20° C. In an embodiment, the loading method includes allowing the bone repair material added with the one or more growth factors to stand at a constant temperature under vacuum or negative pressure.

In an embodiment, the growth factors include IGSF10 and BMP2, which are loaded on the bone repair material in a 1:1 ratio. Of course, the ratio between the growth factors may be adjusted according to specific experimental conditions.

The present disclosure further provides a bone tissue regeneration product, including a bone repair material and growth factors loaded onto the bone repair material, and the growth factor includes IGSF10.

In an embodiment, the growth factors in the product further include one or more of BMPs, PDGF, EMP and amelogenin.

In an embodiment, the bone repair material is a hydroxyapatite scaffold.

The present disclosure provides a method for preparing a bone tissue regeneration product, including adding growth factors to a bone repair material, allowing the bone repair material added with the growth factors to stand for 0.5-2.5 hours at a constant temperature; the growth factors include IGSF10.

Specifically, the preparation method is as follows: slowly adding the growth factor onto the bone repair material dropwise, transferring the bone repair material added with the growth factor to a thermostatic incubator, and standing for 0.5 to 2.5 hours before use. The temperature of the thermostatic incubator is preferably 25-37° C., 4° C. or −20° C.

The growth factors further include one or more of BMP, PDGF, EMP and amelogenin:

The ratio between the growth factors is adjusted according to the actual experiments.

The ratio of the total mass of the growth factors to the total mass volume of the bone repair material is 0.1:1˜15:1, and the unit is ng:mm³. For example, the ratio may be 0.1:1˜1:1, 1:1˜2:1, 2:1˜5:1, 5:1˜8:1, 8:1˜11:1 or 11:1˜15:1.

The present disclosure further provides a method for repairing a bone defect, including the following operations: administering the bone tissue regeneration product to a patient with a bone defect.

Specifically, the bone tissue regeneration product is administered to a bone defect site of the patient with the bone defect.

The amount of growth factors in the product meets the requirement of a therapeutically effective amount. The “therapeutically effective amount” refers to the amount of growth factors that is effective in treating bone defects, especially periodontal bone defects, jaw bone defects, skull defects, and/or long bone defects.

In an embodiment, the therapeutically effective amount is 1-160 ng. For example, the therapeutically effective amount may be a range, and the range is one of 1-10 ng, 10-20 ng, 20-70 ng, 70-120 ng, and 120-160 ng.

The bone defect refers to a bone shortage caused by various reasons, such as trauma or surgery. Bone defects often result in bone nonunion, delayed union or nonunion, and local dysfunction.

The repairing method further includes a series of routine operations, such as anesthesia, incision of the skin, suture, and stitch removal.

The repairing method further includes treating the defect site with a substance before administering the bone tissue regeneration product.

The repairing method may be used in humans or other mammals.

The embodiments of the present disclosure will be described below through exemplary embodiments. Those skilled in this field can easily understand other advantages and effects of the present disclosure according to contents disclosed by the specification. The present disclosure may also be implemented or applied through other different specific implementation modes. Various modifications or changes may be made to all details in the specification based on different points of view and applications without departing from the spirit of the present disclosure.

Before further describing the specific embodiments of the present disclosure, it is understood that the scope of the present disclosure is not limited to the specific embodiments described below; It is also needed to be understood that the terminology of the disclosure is used to describe the specific embodiments, and not to limit the scope of the disclosure: In the present specification and claims, the singular forms “a”, “an” and “the” include the plural forms, unless specifically stated otherwise.

When the numerical values are given by the embodiments, it is needed to be understood that the two endpoints of each numerical range and any value between the two endpoints may be selected unless otherwise stated. Unless otherwise defined, all technical and scientific terms used in the present disclosure have the same meaning as commonly understood by one skilled in the field. In addition to the specific method, equipment and material used in the embodiments, any method, equipment and material in the existing technology similar or equivalent to the method, equipment and material mentioned in the embodiments of the present disclosure may be used to realize the invention according to the understanding of the existing technology by those skilled in the art, and the present disclosure.

The recombinant IGSF10 protein used in the embodiments of the present disclosure is purchased from Abcam, and the article number is ab166199.

### Embodiment 1 Alkaline Phosphatase Staining and Calcium Releasing Detection

1.1 Dental pulp cells (DPCs) and periodontal ligament cells (PDLCs) are inoculated in a 24-well plate at a density of 5×10⁴/mL and coculture with same concentration of IGSF10 or BMP-2 under osteogenic medium. After 9 d of culture, alizarin red staining is performed to observe the calcium deposition.

1.2 The amount of cells required for each assay (2×10⁵ cells) is obtained. The cells are washed with PBS, resuspended in 500 μL of calcium ion detection buffer and placed on ice. The cells are homogenized by pipetting up and down for several times or using a homogenizer. The samples are centrifuged at 4° C. at the highest speed for 2-5 min to remove any insoluble material. The supernatant is collected and transferred to a filter. Two duplicative samples are prepared. All reagents are restored to room temperature. Reaction wells are set up: standard wells=50 μL standard diluent: Sample wells=1-50 μL sample (the volume is adjusted to 50 μL/well with dH2O). Adding 90 μL of color-developing agent to each well containing standards, samples or controls. Adding 60 μL of calcium detection buffer to each well. Mixing and incubating at room temperature for 5-10 minutes, avoiding light. Measuring with a microplate reader (OD575 nm).

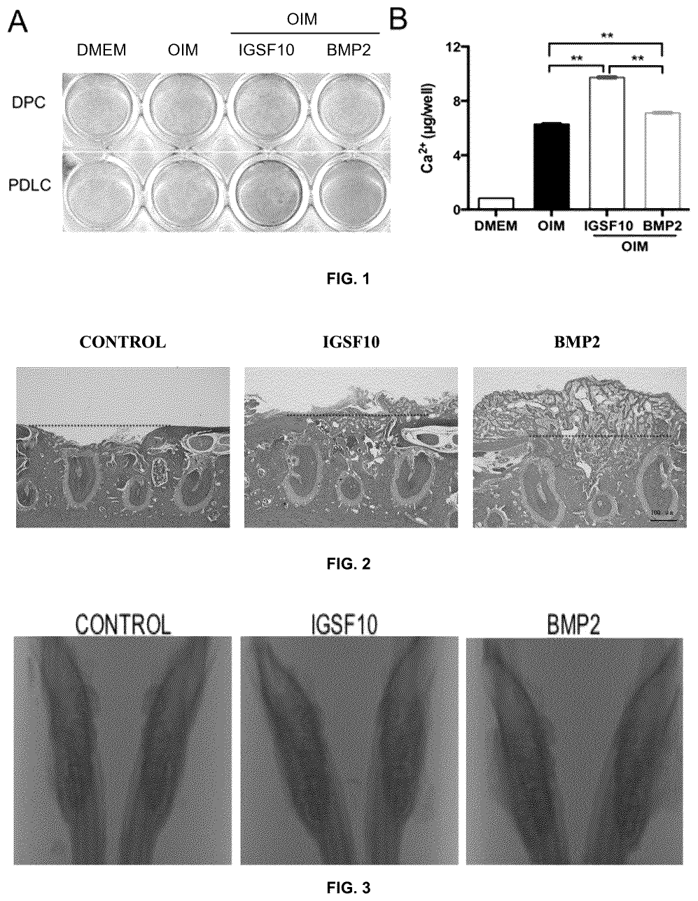

As shown in FIG. 1, alizarin red ARS staining shows that IGSF10 has a strong ability to promote the formation of calcium nodule when the inducing osteogenic solution is present. At the same time, the mineralization promotion effects of IGSF10 and BMP2 are compared, and the results show that IGSF10 is same effective as BMP2 in vitro, which is confirmed by the quantitative results.

### Embodiment 2 Periodontal Defect Experiment in Rats

The surgical region is cleaned and disinfected with 75% ethanol, and all the surgical instruments are thoroughly cleaned and disinfected to minimize contamination. Rats are anesthetized by inhaling a combination of 4% (wt/vol) isoflurane and oxygen. The necessary dose of lidocaine is calculated based on 5 mg/kg after weighing the body and injected subcutaneously on the back. After anesthetized, the supply amount of the nozzle of isoflurane is adjusted to 2.5% (wt/vol) for maintenance. To prevent dryness, eye ointment (lubricant) is applied to the eyes of the rats. After the skin preparation on the surgical region, using povidone-iodine topical antiseptic and sterile saline alternately to scrub outward and spirally to disinfect the skin.

At the inferior margin of the mandible, the skin near the masseter is incised and extended backwards. After the muscle is incised and separated, regions of the mandible and the first molar can be seen. After the distal roots of the first molar as well as the first molar are exposed, a 3×2×1 mm defect is prepared using a No. 4 round drill. Buccal roots and the cementum are removed. IGSF10 and BMP2 are respectively loaded onto collagen gel with the same dose, and transplanted into the defects, respectively; the collagen gel is used as a control group. The muscle is repositioned with absorbable nylon, the wound is sutured, and the skin is cleaned.

As shown in FIGS. 2-3, IGSF10 significantly promotes the regeneration of bone tissue at the defect site, with regular morphology: In the BMP2 group, the stimulation is stronger, with the bone tissue showing expansive growth and the morphology being irregular. Something similar to heterotopic mineralization occurs. It should be noted that, as shown in FIG. 2, the periodontal ligament cells are converted into bone structures even if this area has no bone-related cells such as osteoblast or bone progenitor cells.

### Embodiment 3 Skull Defect Experiment in Mice

Male C57/BL6 mice are selected and weighed, and then anesthetized with isoflurane (5% for anesthetic induction and 1-3% for anesthesia maintenance). The anesthetic status is monitored by pinching the toes of the hind limbs. The respiratory rates are observed at least every 5 minutes. The mice are placed in a prone position, and the surgical plate is kept warm by a circulating warm water blanket. After shaving and disinfecting the skin, the skulls of the mice are disinfected with 70% alcohol. The surgical operators wear clean laboratory gowns, head caps, face masks and sterile gloves. 0.2 mL lidocaine is injected into the incision site under local anesthesia. A 2-cm-long incision is made with a blade along the midline of the skull. The skin and periosteum are bluntly dissected. A round bone defect is prepared on each side of the midline of the skull by using a trephine (outer diameter: 4 mm) under the condition of saline cooling.

In the in-vivo skull defect experiment, IGSF10 and BMP2 with the same dose are loaded onto the hydroxyapatite scaffold. The specific operation method is as follows: according to a ratio 1.3:1 (ng:mm³) of the total mass of IGSF10 and BMP2 to the volume of the hydroxyapatite scaffold, respectively. Moreover, for the combination application, the IGSF10 and BMP2 (R&D, USA) are loaded dropwise onto the hydroxyapatite scaffold at a ratio of 1:1 using a pipettor. The hydroxyapatite scaffold loaded with IGSF10 and BMP2 are transferred to a conventional incubator and allowed to stand for 2 hours, and the solution is loaded into the hydroxyapatite scaffold through the siphoning effect of the porous structure of the hydroxyapatite scaffold for subsequent in vivo experiments. After putting differently processed materials into the skull defect, the wounds are sutured with 5-0 non-absorbable suture materials. The sutures are removed 10-14 days after surgery. All procedures are carried out under aseptic conditions. 8 weeks after surgery, samples are collected and fixed with 4% paraformaldehyde for 2 days, then micro-CT detection is performed.

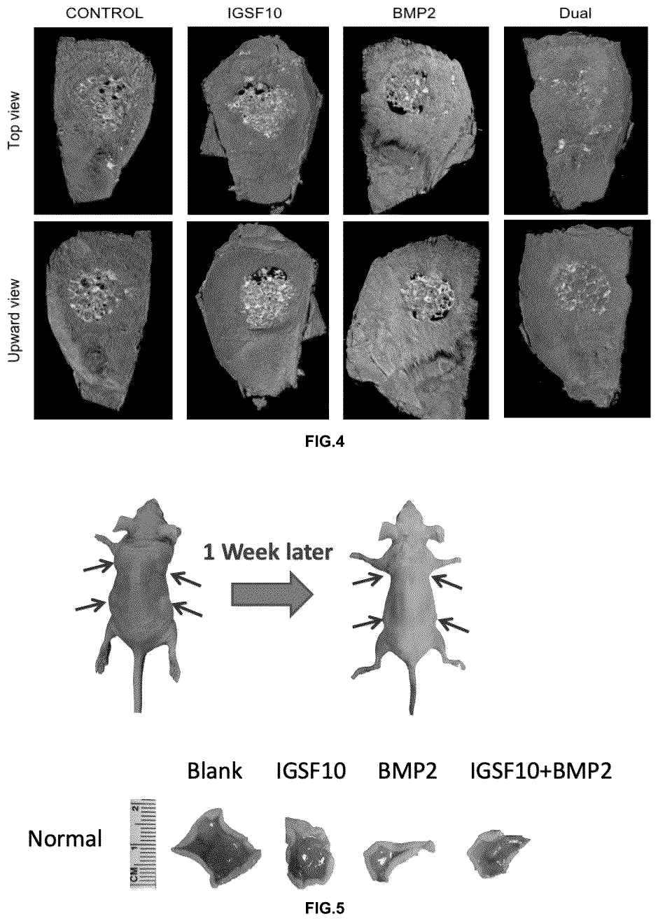

As shown in FIG. 4, IGSF10 and BMP2 with the same dose are loaded on the hydroxyapatite scaffold, both of them can effectively stimulate the formation of new bone. The results of the combined loading show that the skull defect site is completely replaced by new bone, suggesting that IGSF10 greatly improves the osteogenic effect of BMP2.

### Embodiment 4 Subcutaneous Heterotopic Osteogenesis in Nude Mice

Six-week-old BALB/C female nude mice are selected and weighed, and then anesthetized with isoflurane (5% for anesthetic induction and 1-3% for anesthesia maintenance). The anesthetic status is monitored by pinching the toes of the hind limbs. The respiratory rates are observed at least every 5 minutes. The mice are placed in a prone position, and the surgical plate is kept warm by a circulating warm water blanket. The surgical operators wear clean laboratory gowns, head caps, face masks and sterile gloves.

Implants are prepared under sterile conditions: The matrix glue is mixed with rat bone marrow stem cells (BMSCs) on ice to form a suspension with a final density of 2×10⁶ cells/ml. The implants are divided into four groups (as shown in FIG. 5), and growth factors are respectively added to the suspension and mixed thoroughly.

The skin on the back of nude mice is sterilized, 1 ml syringe is used to extract the mixed grafts, and 300 ul implants are injected subcutaneously on the back of nude mice, and the cotton is used to press for 5 seconds. The transplantation is considered to be successful if the transplant site does bleed and the implants do not flow out. The successfully transplanted nude mice are placed in the cage for a few moments for observation, and then transplanted again if the implants fall off. One week later, the nude mice are executed by cervical dislocation, and the implants are fixed in 4% paraformaldehyde buffer.

As shown in FIG. 5, the addition of IGSF10 can promote a more shapely and rigid implant at an early stage.

### Embodiment 5 Cell Spreading Experiments

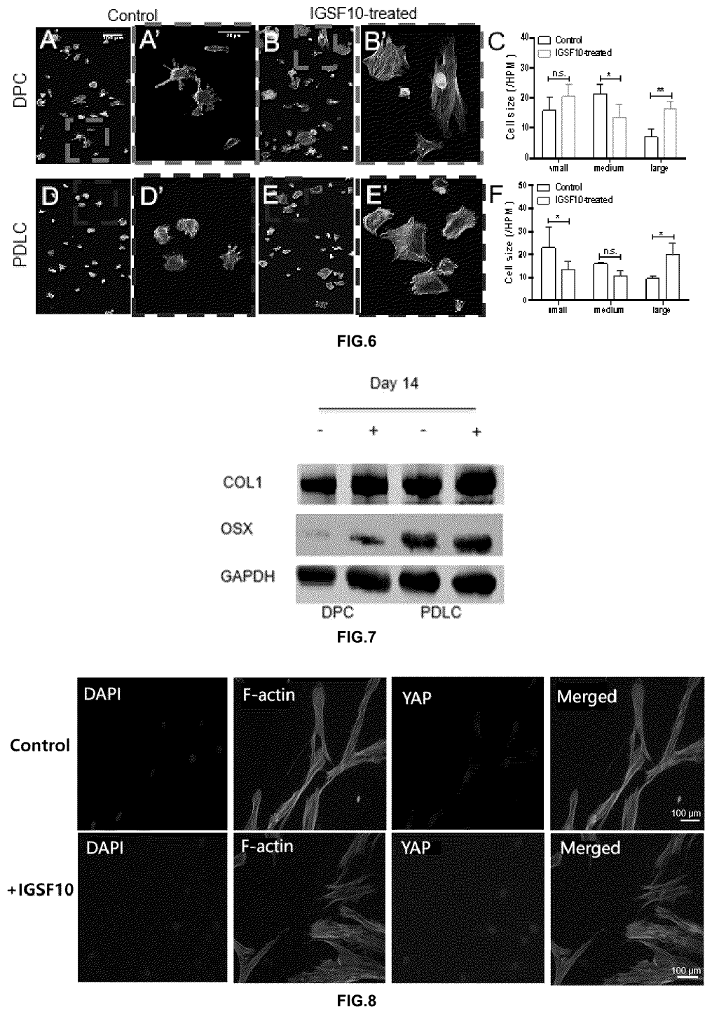

Dental pulp cells (DPCs) and periodontal ligament cells (PDLCs) are inoculated in confocal dishes at a density of 5×10⁴ cells/ml, respectively, and experiments of each type of cell are then divided into 2 groups, i.e., (1) IGSF10-treated group: IGSF10 is added to the medium; and (2) Control group: an equal amount of sterile PBS buffer is added to the medium. After 4 hours, the cells are washed twice with PBS, fixed with 4% paraformaldehyde for 20 min, and stained with 5 ug/ml FITC-phalloidin at room temperature for 30 min. Cells are then washed twice with PBS, and the nuclei of the cells are stained with DAP for 10 min. Cells are washed with PBS, then the excess water is sucked off, and the cells are observed under a confocal microscope. As shown in FIG. 6, the addition of IGSF10 to DPCs and PDLCs can significantly promote cell spreading and migration, which are the earliest behavior of osteogenic differentiation for cells, as proven by a large number of literatures.

### Embodiment 6 Expression Assay of Cellular Osteogenic Indicator Protein

DPCs and PDLCs are inoculated in 6-well plates at a density of 5×10⁴ cells/ml and cultured in osteogenic induction medium, with IGSF10 added to the experimental group, and an equal amount of PBS buffer added to the control group. The medium is changed every 3 days. After 14 days of culture, the original medium is discarded, and the cells are washed three times using the pre-cooled PBS buffer. Appropriate amount of the RIPA cell lysis buffer, protease inhibitors and PMSF in the ratio of 100:1:1 are added to the wells, and the cells are left to lyse for 15 minutes. The supernatant is collected after centrifugation.

The BCA method is used for protein quantification. After determining the protein concentration, protein samples are added to each lane for electrophoresis. After the process is completed, the transfer is performed under ice bath conditions for 40 minutes. Following the transfer, the membrane is washed three times with TBST on a shaker. Then, 5% BSA is added for blocking at room temperature. The primary antibody is diluted at a ratio of 1:1000 and added into the antibody incubation box, ensuring the PVDF membrane is submerged, and incubated overnight at 4° C. on a shaker. On the next day, after washing, the secondary antibody is incubated, and the membrane is developed using the ECL method followed by image processing. As shown in FIG. 7, IGSF10 promotes the expression of osteogenesis-related proteins and bone transcription factors in DPCs and PDLCs, indicating that IGSF10 is capable of promoting the osteogenic differentiation for none-bone cells.

### Embodiment 7 Subcellular Localization Assay of Osteogenesis-Related Transcriptional Co-Activator Proteins

PDLCs are incubated in confocal dishes at a density of 5×10⁴ cells/mL and are divided into the following groups: (1) Control group: an equal volume of PBS buffer is added to the culture medium; (2) IGSF10 group: IGSF10 is added, and cells are treated for 24 hours.

At the end of the experiment, the cells are washed twice with PBS, fixed with 4% paraformaldehyde for 20 minutes, and the primary antibody is diluted to a working solution at a ratio of 1:200, then applied to cover the surface of the cells and incubated overnight at 4° C. in the dark. Depending on the species of the primary antibody; a fluorescently labeled secondary antibody is selected and prepared as a working solution, incubated for 1 hour at room temperature in the dark, and then thoroughly washed with PBS. FITC-phalloidin is added at a concentration of 5 μg/mL for staining at room temperature for 30 minutes. After washing the cells twice with PBS, DAPI is used to stain the cell nuclei for 10 minutes. After washing the cells with PBS and removing excess moisture, the cells are observed under a confocal microscope.

As shown in FIG. 8, the addition of IGSF10 to PDLCs significantly promotes the nuclear translocation of YAP protein. Literature has confirmed that YAP is an important participant in the osteogenic process of bone homeostasis. Acting as a transcriptional co-activator, when YAP translocates to the nucleus, it can bind with osteogenic transcription factors to regulate gene transcription, thereby further affecting the osteogenic differentiation process of the cells.

### Embodiment 8 Investigation of the Osteogenic Mechanism of IGSF10

DPCs and PDLCs are incubated in a six-well plate at a density of 5×10⁴ cells/mL. In this embodiment there are two groups, i.e., (1) IGSF10-treated group: IGSF10 is added to the medium; and (2) control group: an equal amount of sterile PBS buffer is added to the medium.

The treated cells are collected, and the original culture medium is discarded. The cells are then repeatedly washed three times with pre-cooled PBS solution. An appropriate amount of RIPA cell lysis buffer, protease inhibitors, and PMSF are added to the wells at a ratio of 100:1:1, and the cells are left to lyse for 15 minutes. The supernatant is collected after centrifugation.

The BCA method is used for protein quantification. After determining the protein concentration, protein samples are added to each lane for electrophoresis. After the process is completed, the transfer is performed under ice bath conditions for 40 minutes. Following the transfer, the membrane is washed three times with TBST on a shaker. Then, 5% BSA is added for blocking at room temperature. The primary antibody is diluted at a ratio of 1:1000 and added into the antibody incubation box, ensuring the PVDF membrane is submerged, and incubated overnight at 4° C. on a shaker. On the next day, after washing, the secondary antibody is incubated, and the membrane is developed using the ECL method followed by image processing.

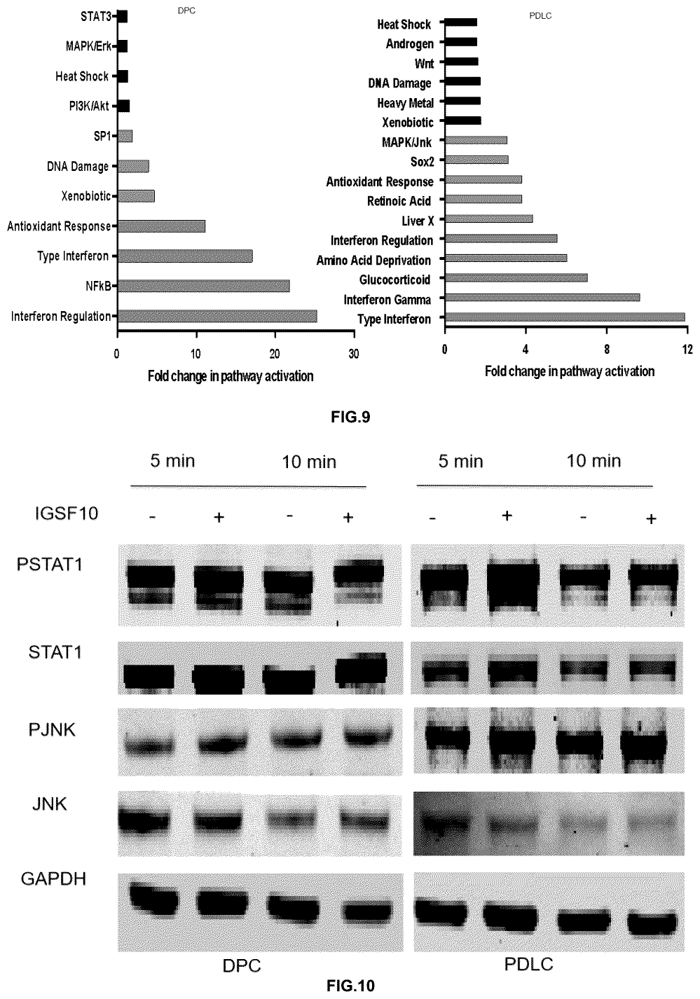

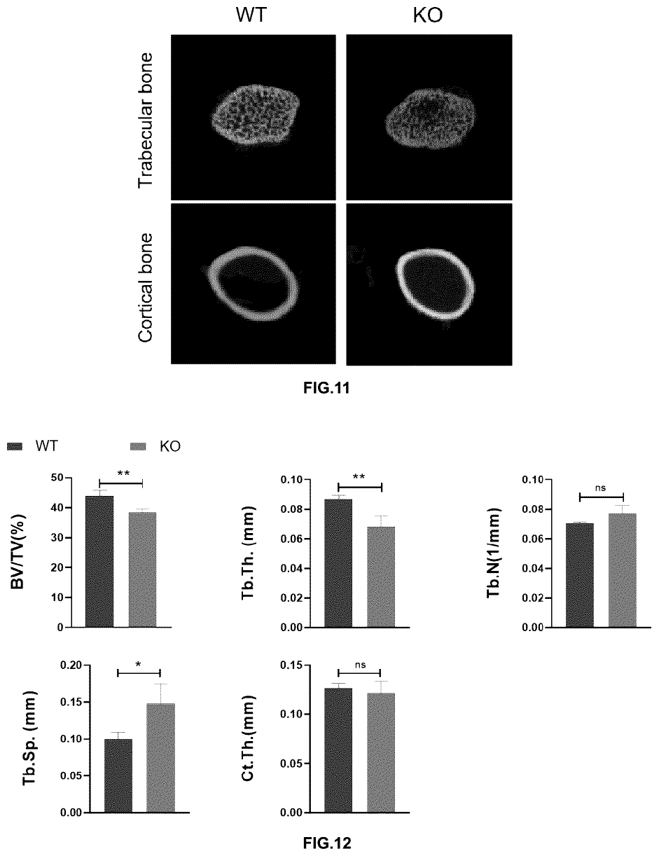

As shown in FIGS. 9-10, it is suggested that IGSF10 may exert its effects through the MAPK/JNK and interferon signaling pathways, rather than the Smad1/5/8 pathways which are predominantly influenced by BMP2. The protein immunoblotting experiment confirms that the expression of pStat1 and pJNK is increased by IGSF10.

### Embodiment 9 Bone Phenotype Assay of IGSF10 Knockout Mouse

Mice with the genotypes IGSF10 flox/flox (wild-type [WT]) and IGSF10 knock out (KO) are selected for the gene knockout of IGSF10, with no fewer than 3 mice of each genotype.

10 ml of sunflower seed oil is sterilized (autoclave, 121° C. for 30 min, cooled to room temperature). 50 mg of Tamoxifen is weighed and placed into 0.5 ml of anhydrous ethanol, dissolved at 55° C. for approximately 1 hour. After complete dissolution, 9.5 ml of sunflower seed oil is added and mixed on a shaker for about 30 minutes.

The syringe is held in the right hand, while the little finger and ring finger of the left hand grasp the mouse's tail, and the other three fingers grasp the mouse's neck, positioning the mouse's head downward. The needle is gently inserted, and the medication is injected at an appropriate dose according to body weight, then the needle is left in place for about 20 seconds before being slowly withdrawn while slightly rotating the needle tip to prevent leakage. This injection is administered once a day for 5 consecutive days. After a week of observation, the mice are executed by cervical dislocation, and the femurs are dissected and fixed in 4% paraformaldehyde for 24 hours before undergoing micro-CT examination.

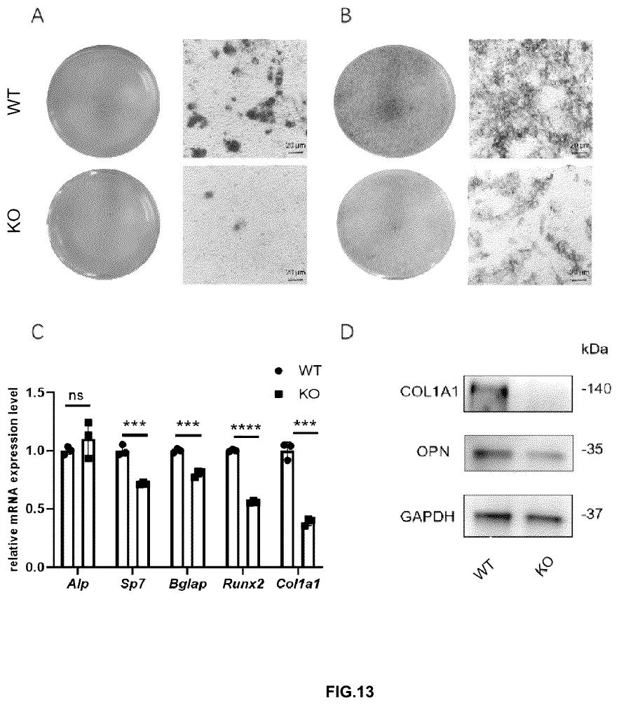

The results, as shown in FIG. 12, indicate that the knockout of the IGSF10 gene can significantly reduce the ratio of bone volume to tissue volume (BV/TV) and the number of trabecular bones (Tb.N) in mice, while markedly increasing the trabecular spacing (Tb.Sp), and both bone mass and bone quantity are significantly decreased. This suggests that IGSF10 is an important regulator of bone homeostasis and has potential practical significance for the prevention and treatment of bone tissue diseases.

### Embodiment 10 Detection of Osteogenic Capacity of BMSC in IGSF10 Knockout Mice

Four-week-old WT and KO mice are executed by cervical dislocation and immersed in 75% ethanol. Subsequently, the skin and inner lining are cut at the location of the mouse's legs, and under sterile conditions, both femurs and tibias are collected and immersed in sterile PBS solution containing antibiotics. The muscles and connective tissues surrounding the femurs and tibias are removed, and the bones are washed three times with PBS solution containing antibiotics. The ends of the femurs and tibias are trimmed with ophthalmic scissors to expose the marrow cavity. The marrow cavity is flushed repeatedly with complete culture medium using a syringe until the bones turn white. The bone marrow fluid is collected, centrifuged, and then inoculated into culture dishes for culture. After 24-48 hours, the suspension is removed, and the medium is changed every three days thereafter until passage is required.

Bone marrow stem cells (BMSCs) are inoculated at a density of 5×10⁴/mL in culture plates and cultured with osteogenic induction medium. After three days, alkaline phosphatase staining and alizarin red staining are performed to observe calcium deposition. For other cells with the same treatment, the original culture medium is discarded, and the cells are washed three times with pre-cooled PBS solution. An appropriate amount of RIPA cell lysis buffer, protease inhibitors and PMSF at a ratio of 100:1:1 is added to the wells, and the cells are left to lyse for 15 minutes. The supernatant is collected after centrifugation.

The BCA method is used for protein quantification. After determining the protein concentration, protein samples are added to each lane for electrophoresis. After the process is completed, the transfer is performed under ice bath conditions for 40 minutes. Following the transfer, the membrane is washed three times with TBST on a shaker. Then, 5% BSA is added for blocking at room temperature. The primary antibody is diluted at a ratio of 1:1000 and added into the antibody incubation box, ensuring the PVDF membrane is submerged, and incubated overnight at 4° C. on a shaker. On the next day, after washing, the secondary antibody is incubated, and the membrane is developed using the ECL method followed by image processing.

As shown in FIGS. 13A-D, the expression levels of osteogenic genes in BMSCs from knockout IGSF10 mice are significantly reduced. ALP and alizarin red staining show a decrease in their calcium nodule formation ability, indicating weakened osteogenic and mineralization capacity.

### Embodiment 11 Periodontitis Experiments on Mice

As mentioned above, inflammation needs to be effectively suppressed before bone formation. The suppression of inflammation by IGSF10 is shown in this embodiment.

Male C57/BL6 mice are weighed and then anesthetized with isoflurane (5% for anesthetic induction and 1-3% for anesthesia maintenance). The anesthetic status is monitored by pinching the toes of the hind limbs. The respiratory rates are observed at least every 5 minutes. The mice are placed in a prone position, and the surgical plate is kept warm by a circulating warm water blanket. The surgical operators wear clean laboratory gowns, head caps, face masks and sterile gloves.

All mice are randomly assigned to three groups, with at least three mice in each group: a healthy control group, a periodontitis group, and a periodontitis treatment group. The healthy control group is left untreated, while the periodontitis group and periodontitis treatment group are constructed as experimental periodontitis mouse models by using the classical silk ligation method, i.e., tying a 5-0 silk ligature around the neck of the maxillary second molar and locally applying lipopolysaccharide (LPS) solution for inflammatory stimulation. The ligatures are checked every 2 days, and any loose ligatures are immediately retied.

In the periodontitis treatment experiment, IGSF10 is dissolved in sterile PBS buffer and locally applied to the periodontal ligature site the day after ligation. The periodontitis group is treated in the same way with sterile PBS buffer.

At the end of the experiment, the mice are executed by cervical dislocation, and the maxilla on the side of the ligation is dissected in the periodontitis groups, while the healthy control group's maxilla at the same side is dissected. After the maxillae are fixed in 4% paraformaldehyde for 24 hours, micro-CT examination and tissue section staining are performed.

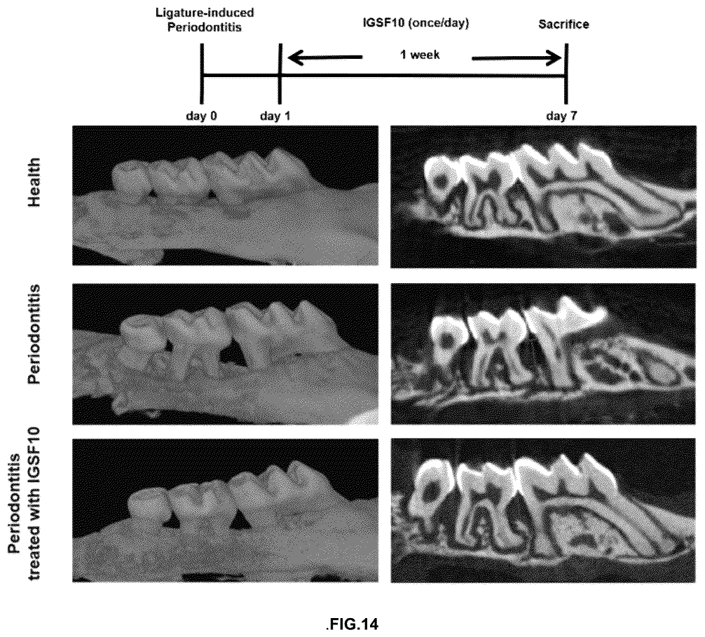

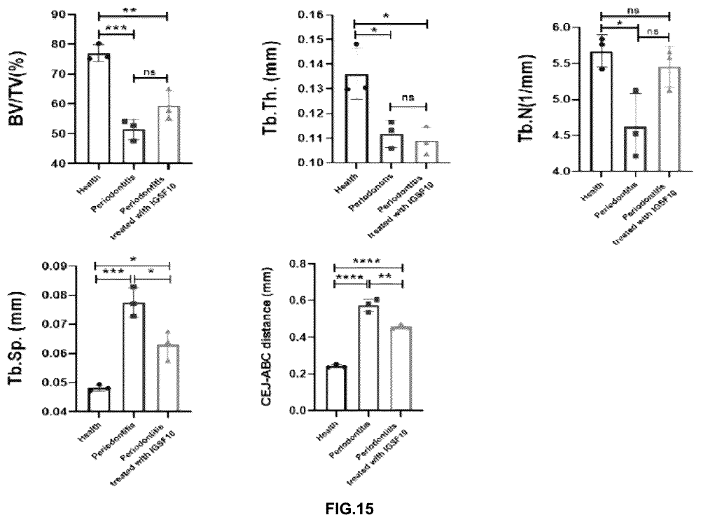

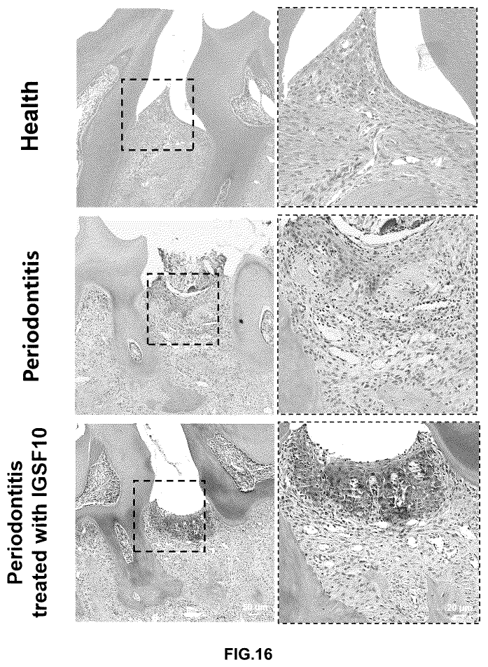

The results, as shown in FIGS. 14-15, indicate that topical periodontal administration of IGSF10 can significantly alleviate the alveolar bone loss in periodontitis mice, reducing the height of alveolar bone loss (measured as the distance from the cemento-enamel junction (CEJ) to the alveolar bone crest (ABC)), and can significantly reduce the inflammatory cell infiltration in periodontal tissues (FIG. 16).

These results suggest that IGSF10, in addition to promoting the regeneration of bone in bone repair, can also significantly inhibit the process of bone loss in inflammatory environments.

The above results show that the use of IGSF10 for bone tissue regeneration and repair can synergize or replace BMP currently in routine clinical use and thereby reducing the dosage of BMP and avoiding adverse reactions. Therefore, promoting the clinical application of IGSF10 is feasible. The above results also show that growth factor IGSF10 is capable of promoting non-bone related cells to produce bones, that is, the present disclosure discovers that IGSF10 can regenerate bone tissue even when there are no osteoblast, bone progenitor cells, or other bone-related cells. In addition, the above results also show that growth factor IGSF10 inhibit the bone loss in inflammatory environments.

The above embodiments are intended to illustrate the disclosed embodiments of the present disclosure and are not understood as restrictions on the present disclosure. In addition, various modifications of the present disclosure, as well as variations of the methods of the present disclosure, will be apparent to those skilled in the art without departing from the scope of the present disclosure. While the disclosure has been described in detail in connection with various specific preferred embodiments thereof, however, it should be understood that the present disclosure should not be limited to these specific embodiments. In fact, various modifications to the present disclosure as apparent to those skilled in the art are intended to be included within the scope of the present disclosure.

### INCORPORATION BY REFERENCE OF SEQUENCE LISTING ON AN XML FILE

The content of the following submission on XML file is incorporated herein by reference in its entirety: a computer-readable form (CRF) of the Sequence Listing (file name: 240333 sequence listing.xml, date recorded: Nov. 1, 2024, size: 4.00 KB).

1. A bone tissue regeneration product, comprising a bone repair material and one or more growth factors loaded onto the bone repair material, wherein the one or more growth factors include growth factor IGSF10.

2. The bone tissue regeneration product according to claim 1, wherein the amino acid sequence of the growth factor IGSF10 is set forth in SEQ ID NO. 1.

3. The bone tissue regeneration product according to claim 1, wherein the one or more growth factors in the product further include one or more of BMP, PDGF, EMP, and amelogenin.

4. The bone tissue regeneration product according to claim 1, wherein the one or more growth factors in the product further include BMP.

5. The bone tissue regeneration product according to claim 1, wherein the bone tissue regeneration product comprises a periodontal bone defect repair product, a jaw bone defect repair product, and/or a skull defect repair product, and/or a long bone defect repair product.

6. The bone tissue regeneration product according to claim 1, wherein the bone tissue regeneration product is drug, health care product, food, or consumable.

7. The bone tissue regeneration product according to claim 1, wherein the bone repair material is a hydroxyapatite scaffold, a calcium phosphate scaffold, a hydrogel scaffold, or a bioglass scaffold.

8. A method for preparing a bone tissue regeneration product, comprising: adding one or more growth factors to a bone repair material; and allowing the bone repair material added with the one or more growth factors to stand for 0.5-2.5 hours at a constant temperature, wherein the one or more growth factors include IGSF10.

9. The method according to claim 8, further comprising one or more of the following:
1) the one or more growth factors further include one or more of BMP, PDGF, EMP and amelogenin;
2) the constant temperature is 25-37° C., 4° C. or −20° C.;
3) freeze-drying the bone repair material added with the growth factors;
4) allowing the bone repair material added with the one or more growth factors to stand at a constant temperature under vacuum or negative pressure;
5) the ratio of the total mass of the one or more growth factors to the total mass of the bone repair material is 0.1:1˜15:1.

10. A method for repairing a bone defect, comprising the following operations: administering a bone tissue regeneration product to a bone defect site, wherein the bone tissue regeneration product comprises a bone repair material and one or more growth factors loaded onto the bone repair material, wherein the one or more growth factors include growth factor IGSF10.

11. The method for repairing a bone defect according to claim 10, wherein the amino acid sequence of the growth factor IGSF10 is set forth in SEQ ID NO. 1.

12. The method for repairing a bone defect according to claim 10, wherein the one or more growth factors in the product further include one or more of BMP, PDGF, EMP, and amelogenin.

13. The method for repairing a bone defect according to claim 10, wherein the one or more growth factors in the product further include BMP.

14. The method for repairing a bone defect according to claim 10, wherein the bone defect site comprises bone related cells and/or non-bone related cells.

15. The method for repairing a bone defect according to claim 10, wherein the bone defect site comprises non-bone related cells and does not comprise bone related cells.

16. The method for repairing a bone defect according to claim 14, wherein the non-bone related cells comprise dental pulp cells and/or periodontal ligament cells.

17. The method for repairing a bone defect according to claim 10, wherein the bone defect comprises a periodontal bone defect, a jaw bone defect, a skull defect, and/or a long bone defect.

18. The method for repairing a bone defect according to claim 17, wherein the bone defect is a periodontal bone defect.

19. The method for repairing a bone defect according to claim 10, further comprising administering the bone tissue regeneration product to an inflammatory tissue microenvironment.

20. The method for repairing a bone defect according to claim 10, comprising: administering the bone tissue regeneration product containing an effective amount of the one or more growth factors to a bone defect site.


SEQUENCE LISTING (1 sequence)

SEQ ID NO: 1 - protein, 99 aa, synthetic construct
SAFISPQGFMAPFGSLTLNMTDQSGNEANMVCSIQKPSRTSPIAFTEENDYIVLNTSFSTFLVCNIDYGHIQPVWQILALYSDSPLILERSHLLSETPQ